(19) United States
(12) Patent Application Publication
(10) Pub. No.: US 2023/0364365 A1
(43) Pub. Date: Nov. 16, 2023

(54) SYSTEMS AND METHODS FOR USER INTERFACE COMFORT EVALUATION

(71) Applicant: ResMed Pty Ltd, Bella Vista (AU)

(72) Inventor: Michael Christopher HOGG, Sydney (AU)

(21) Appl. No.: 18/195,294

(22) Filed: May 9, 2023

Related U.S. Application Data
(60) Provisional application No. 63/340,237, filed on May 10, 2022.

(51) Int. Cl. A61M 16/00; G16H 20/40; G06T 7/20

(52) U.S. Cl. CPC A61M 16/0003; A61M 16/0051; G16H 20/40; G06T 7/20; G06T 2207/30201

(57) ABSTRACT

A method and system for determining a comfort score to evaluate an interface to be worn on a face of a user of a respiratory therapy device. Facial features of the user are determined based on a facial image. Facial feature data from a user population and a corresponding set of interface dimensional data from interfaces used by the user population is stored. Operational data of respiratory therapy devices used by the user population with the interfaces is stored. A comfort score for the interface is determined via an evaluation tool. The evaluation tool determines the comfort score based on the facial features of the user, the output of a simulator simulating the interface on the plurality of facial feature data, and the operational data. The comfort score is displayed on a display to assist a user in selection of an interface with the best comfort.

### CROSS-REFERENCE TO RELATED APPLICATION

This application claims benefit of, and priority under 35 U.S.C. § 119, to U.S. Provisional Patent Application No. 63/340,237, filed May 10, 2022. The contents of that application are hereby incorporated by reference in their entirety.

### TECHNICAL FIELD

The present disclosure relates generally to systems and methods for performing respiratory therapy, and more particularly, to systems and methods for evaluating and improving the comfort of a user wearing a respiratory therapy interface.

### BACKGROUND

A range of respiratory disorders exist. Certain disorders may be characterized by particular events, such as apneas, hypopneas, and hyperpneas. Obstructive Sleep Apnea (OSA), a form of Sleep Disordered Breathing (SDB), is characterized by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected subject to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem.

Other sleep related disorders include Cheyne-Stokes Respiration (CSR), Obesity Hyperventilation Syndrome (OHS) and Chronic Obstructive Pulmonary Disease (COPD). COPD encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors.

Continuous Positive Airway Pressure (CPAP) therapy has been used to treat Obstructive Sleep Apnea (OSA). Application of continuous positive airway pressure acts as a pneumatic splint and may prevent upper airway occlusion by pushing the soft palate and tongue forward and away from the posterior oropharyngeal wall.

Non-invasive ventilation (NIV) provides ventilatory support to a user through the upper airways to assist the user in taking a full breath and/or maintain adequate oxygen levels in the body by doing some or all of the work of breathing. The ventilatory support is provided via a user interface. NIV has been used to treat CSR, OHS, COPD, and Chest Wall disorders. In some forms, the comfort and effectiveness of these therapies may be improved. Invasive ventilation (IV) provides ventilatory support to users that are no longer able to effectively breathe themselves and may be provided using a tracheostomy tube.

A treatment system may comprise a respiratory therapy device, an air circuit, a humidifier, a patient interface, and data management. An interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a user. Depending upon the therapy to be applied, the interface may form a seal, e.g., with a region of the user's face, to facilitate the delivery of gas at a pressure at sufficient variance with ambient pressure to effect therapy, e.g., at a positive pressure of about 10 cm H2O relative to ambient pressure. For other forms of therapy, such as the delivery of oxygen, the interface may not include a seal sufficient to facilitate delivery to the airways of a supply of gas at a positive pressure of about 10 cm H2O. Treatment of respiratory ailments by such therapy may be voluntary, and hence user may elect not to comply with therapy if they find devices used to provide such therapy uncomfortable, difficult to use, expensive and/or aesthetically unappealing.

The design of an interface presents a number of challenges. The face has a complex three-dimensional shape. The size and shape of noses varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. For example, masks designed solely for aviators, masks designed as part of personal protection equipment (e.g., filter masks), SCUBA masks, or for the administration of anesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided user comply with therapy. Obtaining an interface allows a user to engage in treatment such as positive pressure therapy. Users seeking their first interface or a new interface to replace an older interface, typically consult a durable medical equipment provider to determine a recommended interface size based on measurements of the user's facial anatomy, which are typically performed by the durable medical equipment provider. If a mask is uncomfortable, or difficult to use a user may not comply with therapy. Since it is often recommended that a patient regularly wash their mask, if a mask is difficult to clean (e.g., difficult to assemble or disassemble), user may not clean their mask and this may impact on patient compliance. In order for the air pressure therapy to effective, not only must comfort be provided to a user in wearing the mask, but a solid seal must be created between the face and the mask to minimize air leaks.

Interfaces, as described above, may be provided to a user in various forms, such as a nasal mask or full-face mask/oro-nasal mask (FFM) or nasal pillows mask, for example. Such interfaces are manufactured with various dimensions to accommodate a specific user's anatomical features in order to facilitate a comfortable interface that is functional to provide, for example, positive pressure therapy. Such interface dimensions may be customized to correspond with a particular facial anatomy or may be designed to accommodate a population of individuals that have an anatomy that falls within predefined spatial boundaries or ranges. However, in some cases masks may come in a variety of standard sizes from which a suitable one must be chosen.

In this regard, sizing an interface for a user is typically performed by a trained individual, such as a Durable Medical Equipment (DME) provider or physician. Typically, a user needing an interface to begin or continue positive pressure therapy would visit the trained individual at an accommodating facility where a series of measurements are made in an effort to determine an appropriate interface size from standard sizes. An appropriate size is intended to mean a particular combination of dimensions of certain features, such as the seal forming structure, of an interface, which provide adequate comfort and sealing to effectuate positive pressure therapy. Sizing in this way is not only labor intensive but also inconvenient. The inconvenience of taking time out of a busy schedule or, in some instances, having to travel great distances is a barrier to many patients receiving a new or replacement interface and ultimately a barrier to receiving treatment. This inconvenience prevents users from receiving a needed interface and from engaging in respiratory therapy. Nevertheless, selection of the most appropriate type of interface is important for treatment quality and compliance.

Many styles and models of user interface exist, allowing a user to select a particular user interface that is comfortable and effective. Generally, user interface fitting is a lengthy procedure conducted by a medical provider trained to fit user interfaces. While such fitting procedures can be useful to establish a baseline, once the user goes home and attempts to use the respiratory therapy system, it is up to the user to ensure the user interface is properly fit to the user's face. Therefore, it can be beneficial to provide a user with the tools necessary to evaluate and/or improve the fit of the user interface themselves. Additionally, if an in-person visit to a medical provider is undesirable or otherwise contraindicated, it can be useful to have a way to fit a user interface without requiring an in-person visit to the medical provider's office. Also, the ability for a user to self-perform user interface fit evaluation can enable respiratory therapy systems to be distributed where access to a medical provider trained in respiratory therapy fitting is limited. Even if proper fit is established, actual comfort to the user cannot be currently evaluated, thus certain users still do not adhere to therapy even if technically, the selected interfaces fit the face of the user.

There is a need for a system that allows for determining comfort of an interface for a respiratory therapy device in relation to facial features of an individual user. There is another need for an application that displays interfaces that may fit the facial features of an individual user and rank the interfaces by a comfort score. There is a further need for an application that employs machine learning trained on simulations of interfaces in relation to facial data to determine a comfort score based on the input of facial image data and data relating to a selected interface.

### SUMMARY

According to some implementations of the present disclosure, a method to evaluate an interface to be worn on a face of a user of a respiratory therapy device is disclosed. A facial image of the user is stored in a storage device. Facial features of the user are determined based on the facial image. Facial feature data from a user population and a corresponding plurality of interface dimensional data from interfaces used by the user population are stored in one or more databases. Operational data of respiratory therapy devices used by the user population with the of interfaces is stored in one or more databases. A comfort score for the interface is determined via an evaluation tool. The evaluation tool determines the comfort score based on the facial features of the user, the output of a simulator simulating the interface on the facial feature data, and the operational data. The comfort score is displayed on a display.

A further implementation of the example method is where the interface is a mask. Another implementation is where the evaluation tool includes a machine learning model outputting the comfort score based on the facial image data and dimensional data of the interface. Another implementation is where the method includes training the machine learning model by comparing comfort scores determined from the simulator simulating the plurality of interfaces based on the interface dimensional data worn on faces of the user population based on the facial feature data, with comfort scores provided from the user population. Another implementation is where the comfort scores provided from the user population are determined based on at least one of operational data of the respiratory therapy devices, the facial features data, or subjective responses of the user population derived from answers of a survey. Another implementation is where the simulator models the interfaces worn on faces of the user population with finite element analysis. Another implementation is where the dimensional data of the interfaces is computer aided design (CAD) data. Another implementation is where the simulation simulates pushing the interfaces into the simulated faces until a seal is between the simulated faces and the simulated interface is obtained, the pressurization of the simulated interfaces, and a resulting gap between the simulated interfaces and the simulated faces. Another implementation is where the simulator outputs interface deformation, contact gaps between skin of the simulated faces and cushions of the interfaces, contact pressure/shear on skin of the simulated face, skin deformation of the simulated faces, and stress/strain in the cushions of the interfaces. Another implementation is where the selected interface is one of the plurality of interfaces and one of a plurality of sizes of each of the plurality of interfaces. Another implementation is where the selected interface is one of a subset of interfaces selected from the plurality of interfaces that fit the face of the user. Another implementation is where the displaying includes displaying the subset of interfaces and associated comfort scores. Another implementation is where the display is on a mobile user device. Another implementation is where the evaluation tool accepts demographic data of the user to determine the comfort score. Another implementation is where the respiratory therapy device is configured to provide one or more of a Positive Airway Pressure (PAP) or a Non-invasive ventilation (NIV). Another implementation is where the operational data from the respiratory therapy devices includes data to determine leaks in the operation of the respiratory therapy devices. Another implementation is where the method includes scanning the face of the user via a mobile device including a camera to provide the facial image. Another implementation is where the mobile device includes a depth sensor. The camera is a 3D camera, and the facial features are three-dimensional features derived from a meshed surface derived from the facial image. Another implementation is where the facial image is a two-dimensional image including landmarks. The facial features are three-dimensional features derived from the landmarks. Another implementation is where the facial image is one of a plurality of two-dimensional facial images. The facial features are three-dimensional features derived from a 3D morphable model adapted to match the facial images. Another implementation is where the facial image includes landmarks relating to at least one facial dimension. Another implementation is where the facial dimension includes at least one of face height, nose width, and nose depth. Another implementation is where the method further includes determining a predicted leak of the interface via the evaluation tool.

Another disclosed example is a system including a control system comprising one or more processors. The system includes a memory having stored machine readable instructions. The control system is coupled to the memory. The above methods are implemented when the machine executable instructions in the memory are executed by at least one of the one or more processors of the control system. Another disclosed example is a system for evaluating a selected user interface. The system includes a control system configured to implement the above referenced methods.

Another disclosed example is a computer program product comprising instructions which, when executed by a computer, cause the computer to carry out the above methods. Another implementation of the computer program product is where the computer program product is a non-transitory computer readable medium.

According to some implementations of the present disclosure, a system for evaluating a selected interface worn by a user using a respiratory therapy device is disclosed. The system includes a storage device for storing facial image data of the user. The system has one or more databases for storing facial feature data from a user population and a corresponding plurality of interface dimensional data from interfaces used by the user population. The databases store operational data of respiratory therapy devices used by the user population with the interfaces. A facial comfort interface evaluation tool is coupled to the storage device. The evaluation tool outputs a comfort score of the interface based on analysis of the facial image data of the user, the output of a simulator simulating the interface on the facial feature data, and the operational data. The system includes a display to display the comfort score of the interface.

A further implementation of the example system is where the interface is a mask. Another implementation is where the evaluation tool includes a machine learning model outputting the comfort score based on the facial image data and dimensional data of the interface. Another implementation is where the machine learning model is trained from comparing comfort scores determined from the simulator simulating the interfaces based on the interface dimensional data worn on faces of the user population based on the facial feature data, with comfort scores provided from the user population. Another implementation is where the comfort scores provided from the user population are determined based on at least one of operational data of the respiratory therapy devices, the facial features data, or subjective responses of the user population derived from answers of a survey. Another implementation is where the simulator models the plurality of interfaces worn on faces of the user population with finite element analysis. Another implementation is where the dimensional data of the interfaces is computer aided design (CAD) data. Another implementation is where the simulation simulates pushing the interfaces into the simulated faces until a seal is between the simulated faces and the simulated interface is obtained, the pressurization of the simulated interfaces, and a resulting gap between the simulated interfaces and the simulated faces. Another implementation is where the simulator outputs interface deformation, contact gaps between skin of the simulated faces and cushions of the interfaces, contact pressure/shear on skin of the simulated face, skin deformation of the simulated faces, and stress/strain in the cushions of the interfaces. Another implementation is where the selected interface is one of the plurality of interfaces and one of a plurality of sizes of each of the interfaces. Another implementation is where the selected interface is one of a subset of interfaces selected from the plurality of interfaces that fit the face of the user. Another implementation is where the display displays the subset of interfaces and associated comfort scores. Another implementation is where the display is on a mobile user device. Another implementation is where the evaluation tool accepts demographic data of the user to determine the comfort score. Another implementation is where the respiratory therapy device is configured to provide one or more of a Positive Airway Pressure (PAP) or a Non-invasive ventilation (NIV). Another implementation is where the operational data from the respiratory therapy devices includes data to determine leaks in the operation of the respiratory therapy devices. Another implementation is where the stored facial image data is determined from a scan from a mobile device including a camera. Another implementation is where the mobile device includes a depth sensor. The camera is a 3D camera. The facial features are three-dimensional features derived from a meshed surface derived from the facial image. Another implementation is where the facial image is a two-dimensional image including landmarks. The facial features are three-dimensional features derived from the landmarks. Another implementation is where the facial image is one of a plurality of two-dimensional facial images. The facial features are three-dimensional features derived from a 3D morphable model adapted to match the facial images. Another implementation is where the facial image includes landmarks relating to at least one facial dimension. Another implementation is where the facial dimension includes at least one of face height, nose width, and nose depth. Another implementation is where the evaluation tool outputs a predicted leak of the interface.

According to some implementations of the present disclosure, a method of training a machine learning model to output a comfort score for an interface worn by a user is disclosed. Dimensional data for a plurality of interfaces for a respiratory therapy device is collected. Facial data from a plurality of faces of users wearing the plurality of interfaces is collected. A comfort score for each of the plurality of interfaces worn by users is determined. The plurality of interfaces worn on faces of the user population is simulated based on dimensional data of the plurality of interfaces and facial dimensional data derived from the facial data of the plurality of faces. A training data set of the dimensional data of the plurality of interfaces and the facial dimension data is created. The machine learning model is adjusted by providing the training data set and the simulation to predict a comfort score for each face and worn interface, and comparing the predicted comfort score with the associated determined comfort score.

A further implementation of the example method is where the comfort scores provided from the user population are determined based on at least one of operational data of the respiratory therapy devices, the facial features data, or subjective responses of the user population derived from answers of a survey. Another implementation is where the simulator models the interfaces worn on faces of the user population with finite element analysis. Another implementation is where the dimensional data of the interfaces is computer aided design (CAD) data. Another implementation is where the simulation simulates pushing the interfaces into the simulated faces until a seal is between the simulated faces and the simulated interface is obtained, the pressurization of the simulated interfaces, and a resulting gap between the simulated interfaces and the simulated faces. Another implementation is where the simulator outputs interface deformation, contact gaps between skin of the simulated faces and cushions of the interfaces, contact pressure/shear on skin of the simulated face, skin deformation of the simulated faces, and stress/strain in the cushions of the interfaces.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 is a block diagram of a data collection system that collects data to determine the comfort score of user interfaces for a respiratory therapy device, according to some implementations of the present disclosure.

FIG. 4A is an example facial scan that shows different landmark points to identify facial dimensions for mask sizing;

FIG. 4B is a view of the facial scan in FIG. 4A that shows different landmark points to identify a first facial measurement;

FIG. 4C is a view of the facial scan in FIG. 4A that shows different landmark points to identify a second facial measurement;

FIG. 4D is a view of the facial scan in FIG. 4A that shows different landmark points to identify a third facial measurement;

FIG. 5 is a block diagram of a mobile device application and a Cloud based application that allows the determination of interface recommendations for a user;

FIG. 6 is a block diagram of the process of obtaining data for a simulation of interface types for the application in FIG. 5;

FIG. 7 is a block diagram of the process of training and using machine learning models for determining a comfort score for different interfaces for the application in FIG. 5;

FIG. 8 is a screen image of a user interface of a mask selection application allowing a user to display comfort information for mask selections; and

FIG. 9 is a flow diagram for the routine for determining comfort scores for different interfaces.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

Certain aspects and features of the present disclosure relate to evaluating and improving the comfort of a user interface (e.g., a filtering facemask or a user interface, also known as a patient interface, of a respiratory device). Sensor data from one or more sensors of a user device (e.g., portable user device, such as a smartphone) can be leveraged to help a user ensure that a wearable user interface (e.g., user interface of a respiratory device) is properly fit to the user's face. The sensor(s) can collect data about the user's face: i) before donning of the user interface; ii) while the user interface is worn; and/or iii) after removal of the user interface. A face mapping can be generated, identifying one or more features of the user's face. The sensor data and face mapping can then be leveraged to identify characteristics associated with the comfort of the user interface, which are usable to generate output feedback to evaluate and/or improve the comfort of the user interface. As will be explained, the comfort fit system evaluates facial data obtained from a facial scan, subjective patient data obtained from existing user data and user feedback from wearing an interface, and models of available interfaces. The example comfort fit system utilizes a machine learning model that takes the face shape of the user as an input, a subset of interfaces and corresponding interface model data and then returns a mask recommendation. This recommendation includes the best sized interfaces as well as a predicted comfort level. When presented to the user, appropriate interfaces evaluated by the system are ranked in order of the predicted comfort level.

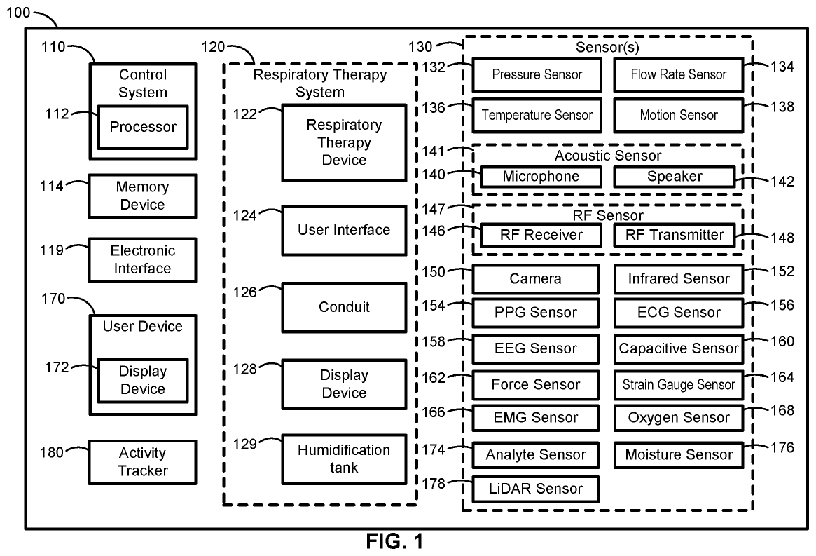

Referring to FIG. 1, a system 100, according to some implementations of the present disclosure, is illustrated. The system 100 includes a control system 110, a memory device 114, an electronic interface 119, one or more sensors 130. In some implementations, the system 100 further optionally includes a respiratory therapy system 120, a user device 170, and an activity tracker 180. In some cases, some or most of system 100 can be implemented as a user device 170 (e.g., the control system 110, processor 112, memory device 114, electronic interface 119, display device 172, and one or more sensors 130 can be implemented in a user device 170, such as a smartphone, smartwatch, tablet, computer, or the like).

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is illustrated in FIG. 1, the control system 110 can include any number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a respiratory therapy device 122 of the respiratory therapy system 120, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subj ective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy device 122 (referred to herein as respiratory device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance of a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

As used herein, in some cases, the term user interface 124 is further inclusive of a device that engages a portion of the user's face and filters air being inhaled by and/or exhaled by the user, whether or not it is coupled to a respiratory therapy device 122. For example, the term user interface 124 can include a user interface 124 that is associated with a respiratory therapy system 120 or a user interface 124 that is a facemask or surgical mask used to filter air.

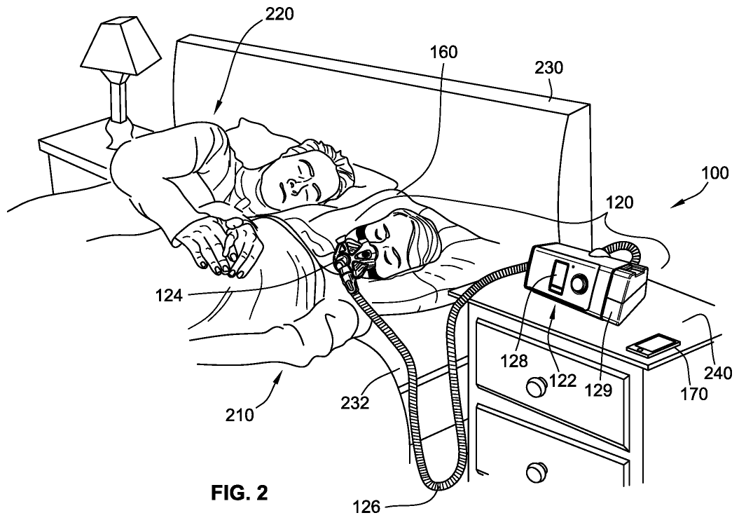

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps forming, for example, a headgear for aiding in positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to teeth of the user, a mandibular repositioning device, etc.).

The conduit 126 (also referred to as an air circuit or tube) allows the flow of air between two components of a respiratory therapy system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score and/or a therapy score, also referred to as a myAirTM score, such as described in WO 2016/061629, which is hereby incorporated by reference herein in its entirety; the current date/time; personal information for the user 210; etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152 (e.g., a passive infrared sensor or an active infrared sensor), a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, sensor data, such as image data, audio data, rangefinding data, contour mapping data, thermal data, physiological data, ambient data, and the like. The sensor data can be used by the control system 110 to identify characteristics associated with a current fit of the user interface 124.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in WO 2012/012835, which is hereby incorporated by reference herein in its entirety. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a localized or average skin temperature of the user 210, a localized or average temperature of the air flowing from the respiratory device 122 and/or through the conduit 126, a localized or average temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof. In some cases, the temperature sensor 136 is a non-contact temperature sensor, such as an infrared pyrometer.

The microphone 140 outputs audio data that can be stored in the memory device 114 and/or analyzed by the processor 112 of the control system 110. The audio data generated by the microphone 140 is reproducible as one or more sound(s) (e.g., sounds from the user 210). The audio data form the microphone 140 can also be used to identify (e.g., using the control system 110) or confirm characteristics associated with a user interface, such as the sound of air escaping a valve. The microphone 140 can be coupled to or integrated in the respiratory device 122, the user interface 124, the conduit 126, the user device 170.

The speaker 142 outputs sound waves that are audible to a user of the system 100 (e.g., the user 210 of FIG. 2). The speaker 142 can be used, for example, to provide audio feedback, such as to indicate how to manipulate the user device 170 to achieve desirable sensor data, or to indicate when the collection of sensor data is sufficiently complete. In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the user 210 or the bed partner 220 (FIG. 2). Based at least in part on the data from the microphone 140 and/or the speaker 142, the control system 110 can determine location information pertaining to the user and/or the user interface 124 (e.g., a location of the user's face, a location of features on the user's face, a location of the user interface 124, a location of features on the user interface 124), physiological parameters (e.g., respiration rate), and the like. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine location information pertaining to the user 210 and/or user interface 124, and/or one or more of the physiological parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine information associated with the face of the user, the user interface 124, and/or one or more of the physiological parameters described herein. For example, the image data from the camera 150 can be used to identify a location of the user, a localized color of a portion of the user's face, a relative location of a feature on the user interface with respect to a feature on the user's face, or the like. In some implementations, the camera 150 includes a wide angle lens or a fish eye lens. The camera 150 can be a camera that operates in the visual spectrum, such as at wavelengths between at or approximately 380 nm and at or approximately 740 nm.

The infrared (IR) sensor 152 outputs infrared image data reproducible as one or more infrared images (e.g., still images, video images, or both) that can be stored in the memory device 114. The IR sensor 152 can be a passive sensor or an active sensor. A passive IR sensor 152 can measure natural infrared emissions or reflections from distant surfaces, such as measuring IR energy radiating from a surface to determine the surface's temperature. An active IR sensor 152 can include an IR emitter that generates an IR signal, which is then received by an IR receiver. Such an active IR sensor 152 can be used to measure IR reflection off and/or transmission through an object. For example, an IR emitter that is a dot projector can project a recognizable array of dots onto a user's face using IR light, the reflections of which can then be detected by an IR receiver to determine ranging data (e.g., data associated with a distance between the IR sensor 152 and a distant surface, such as portion of the user's face) or contour data (e.g., data associated with relative heights features of a surface with respect to a nominal height of the surface)) associated with the user's face.

Generally, the infrared data from the IR sensor 152 can be used to determine information pertaining to the user 210 and/or user interface 124, and/or one or more of the physiological parameters described herein. In an example, the infrared data form the IR sensor 152 can be used to detect localized temperatures on a portion of the user's face or a portion of the user interface 124. The IR sensor 152 can also be used in conjunction with the camera 150, such as to correlate IR data (e.g., temperature data or ranging data) with camera data (e.g., localized colors). The IR sensor 152 can detect infrared light having a wavelength between at or approximately 700 nm and at or approximately 1 mm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte, such as in the exhaled breath of the user 210. The data output by the analyte sensor 174 can be stored in the memory device 114 and used by the control system 110 to determine the identity and concentration of any analytes, such as in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps (e.g., contour maps) of objects such as the user's face, the user interface 124, or the surroundings (e.g., a living space). LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of a user's face, a user interface 124 (e.g., when worn on a user's face), and/or an environment. In a further use, for solid surfaces through which radio waves pass (e.g., radio-translucent materials), the LiDAR may reflect off such surfaces, thus allowing a classification of different type of obstacles. While a LiDAR sensor 178 is described herein, in some cases one or more other ranging sensors can be used instead of or in addition to a LiDAR sensor 178, such as an ultrasonic ranging sensor, an electromagnetic RADAR sensor, and the like.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.). In some implementations, at least one or at least two of the one or more sensors 130 is integrated in and/or coupled to the user device 170.

The user device 170 (FIG. 1) includes a display 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display 172 is generally used to display image(s) including still images, video images, or both. In some implementations, the display 172 acts as a human-machine interface (HMI) that includes a graphic user interface (GUI) configured to display the image(s) and an input interface. The display 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory therapy system 120, and/or the user device 170.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, at least one of the one or more sensors 130, and a user interface 124, but does not include any other components of the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display 172 of the user device 170 (FIG. 1) to manually initiate or terminate the sleep session.

The above described components may be used to collect data on the user in relation to interface use for purposes of evaluating different interfaces based on comfort scores. The evaluation is based on use data relating to the user, facial features of the user, and simulation modeling of different interface options. Thus, the example of the present technology may allow users to more quickly and conveniently obtain a comfortable user interface such as a mask by integrating data gathered from use of respiratory therapy devices in relation to different masks by a user population, facial features of the individual user determined by a scanning process, and mask modeling data. The scanning process allows a user to quickly measure their facial anatomy from the comfort of their own home using a computing device, such as a desktop computer, tablet, smart phone or other mobile device such as the user device 170. The computing device may then receive or generate a recommendation for an appropriate user interface size and type after analysis of the facial dimensions of the user and data from a general user population using a variety of different interfaces.

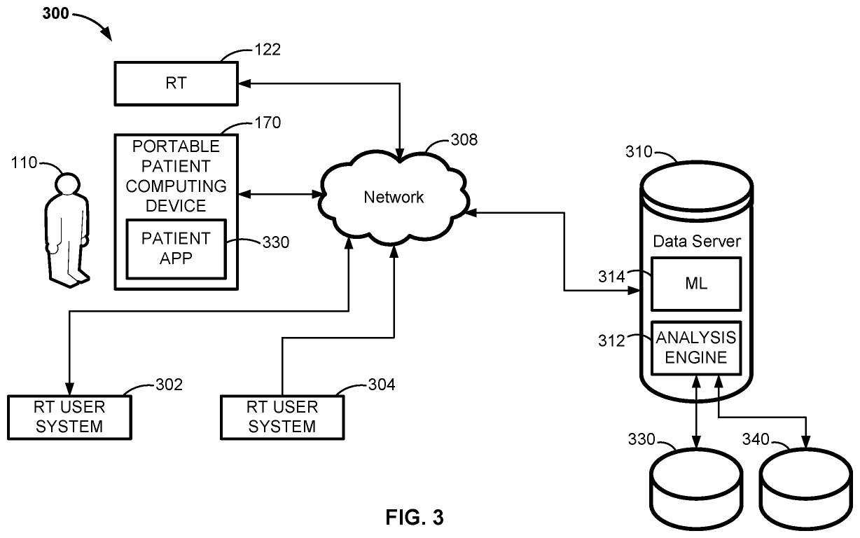

FIG. 3 shows an evaluation system 300 that collects the data relevant to evaluate different interfaces for comfort for a particular user such as the user 110. The evaluation system 300 collects operational data from respiratory therapy systems such as the combination of the respiratory device 122 and the user device 170 in FIG. 1. A population of users of respiratory therapy systems 302 and 304 represents users such as the user 110 that employ respiratory therapy devices that each use interfaces such as masks. The respiratory therapy systems 302 and 304 communicate with a server 310 via a network 308. An interface evaluation engine 312 executed by the server 310 is used to correlate and determine effective mask sizes and types from the individual facial dimensional data and corresponding effectiveness from operational data collected by the respiratory systems 302 and 304 encompassing an entire user population. For example, an effective fit may be evidenced by minimum detected leaks, a high comfort level, maximum compliance with a therapy plan (e.g., mask on and off times, frequency of on and off events, therapy pressure used), number of apneas overnight, AHI levels, pressure settings used on their device and also prescribed pressure settings. This data may be correlated with facial dimensional data or other data based on the facial image of a new user to provide a comfort score for each interface that fits the face of the user. A comfort score may be obtained by the evaluation engine 312 via a trained model executed by a machine learning module 314.

The system 300 may comprise one or more databases. The one or more databases may include a user database 330, and a user interface database 340 and any other database described herein. It is to be understood that in some examples of the present technology, all data required to be accessed by a system or during performance of a method may be stored in a single database. In other examples the data may be stored in two or more separate databases. Accordingly, where there is a reference herein to a particular database, it is to be understood that in some examples the particular database may be a distinct database and in other examples it may be part of a larger database.

In some examples, the user database 330 stores facial features from a user population and a corresponding number of user interfaces used by the user population, and preferably with user subjective data on the performance of the user interface, and operational data of respiratory pressure therapy devices used by the user population with the plurality of corresponding user interfaces. The user interface database 340 stores data on different types and sizes of interfaces, such as masks, that may be available for a new user of a respiratory therapy system. The user interface database 340 may include dimensional data provided by computer aided design (CAD) data for each type of interface for each size interface. The user interface database 340 may also include acoustic signature data of each type of mask that may enable the determination of mask type from audio data collected from respiratory therapy devices. The database 340 may also store subjective data collected from users corresponding with the masks being used and their facial scans to correlate comfort score (or any other scores) with how it actually feels for the user.

For example, the face shape derived from a 2D image or 3D model (whether from a 3D scanner or from converting a 2D image to a 3D model by computational means) may be compared to the geometry of the features of a proposed mask (cushion, conduit, headgear). The difference between the shape and the geometry may be analyzed to determine if there are fit issues that might result in leaks or high contact pressure regions leading to redness/soreness from the contact areas. As explained herein, the data gathered for a population of users, may be combined with other forms of data such as detected leaks to identify the best mask system for a particular face shape (i.e. shape of mouth, nose, cheeks, head etc.).

As will be explained, the server 310 collects the data from multiple users stored in the database 330 and corresponding mask size and type data stored in the interface database 340 to evaluate masks that fit the scanned facial dimensional data collected from the new user. The interface evaluation engine 312 evaluates user interfaces for the user based on a desired maximized comfort based on the stored operational data, facial features of the user, and interface models. The system 300 may be configured to perform a corresponding method of evaluating a user interface. Thus, the system 300 may provide a mask recommendation to a new user by determining what mask has been shown to be optimal for existing users similar in various ways to the new user. The optimal mask may be the mask type, model and/or size that has been shown to be associated with greatest compliance with therapy, lowest leak, fewest apneas, lowest AHI and most positive subjective user feedback, for example. The influence of each of these results in the determination of the optimal mask may be given various different weightings in various examples of the present technology.

In a beneficial embodiment, the present technology may employ an application downloadable from a manufacturer or third-party server to a smart phone or tablet with an integrated camera such as the camera 150 in FIG. 1. When launched, the application may provide visual and/or audio instructions. As instructed, the user (i.e., a user) may stand in front of a mirror, and press the camera button on a user interface. An activated process may then take a series of pictures of the user's face, and then, within a matter of seconds for example, obtain facial dimensions for selection of an interface (based on the processor analyzing the pictures).

Other examples may include identification of three-dimensional facial features from the images. Identification of facial features is sizing based on the “shape” of different features. The shape is described as the near continuous surface of a user's face. In reality, a continuous surface is not possible, but collecting around 10 k-100 k points on the face provides an approximation of the continuous surface of the face. There are several example techniques for collecting facial image data for identifying three-dimensional facial features.

One method may be determining the facial images from a 2D image. In this method, computer vision (CV) and a trained machine learning (ML) model are employed to extract key facial landmarks. For example, OpenCV and DLib libraries may be used for landmark comparison through having a trained number of standard facial landmarks. Once the preliminary facial landmarks are extracted, the derived three-dimensional features must be properly scaled. Scaling involves determining an object such as a coin, credit card or the iris of the user to provide a known scale. For example, Google Mediapipe Facemesh and Iris models may track the iris of a user and scale face landmarks for the purposes of mask sizing. These models contain 468 landmarks of the face and 10 landmarks of the eyes. The iris data is then used to scale other identified facial features.

Another method of determining three-dimensional features may be from facial data taken from a 3D camera with a depth sensor. 3D cameras (such as that on the iPhone X and above) can perform a 3D scan of a face and return a meshed (triangulated) surface. The number of surface points is generally in the order of ˜50 k. In this example, there are 2 types of outputs from a 3D camera such as the iPhone. These are: (a) raw scan data, and (b) a lower resolution blendshape model used for face detection and tracking. The latter includes automatic landmarking, whereas the former does not. The mesh surface data does not require scaling.

Another method is generating a 3D model directly from a 2D image. This involves using a 3D morphable model (or 3DMM) and machine learning to adapt the shape of the 3DMM to match the face in the image. Single or multiple image views are possible from multiple angles and may be derived from a video captured on a digital camera. The 3DMM may be adapted to match the data taken from the multiple 2D images via a machine learning matching routine. The 3DMM may be adapted to account for the shape, pose, and expression shown in the facial image to modify the facial features. Scaling may still be required, and thus detection and scaling of a known object such as an eye feature such as an iris could be used as a reference to account for scaling errors due to factors such as age.

The three-dimensional features or shape data may be used for mask sizing and determination of comfort of masks. One way to match a mask is aligning the identified surfaces of the face with the known surfaces of the proposed mask. The surfaces are then aligned. The alignment may be performed by the nearest iterative closest point (NICP) technique. For mask fitting purposes, a fit score may then be calculated by determining the mean average distances, which is the mean of distances between the closest or corresponding points of the facial features and the mask contact surfaces. A low score corresponds to a good fit. As will be explained, other scores for masks such as a comfort score may be determined with the facial data.

Another method of mask sizing may be to use 3D face scans collected from different users. In this example, 3D data may be collected for over 1,000 users. These users are grouped according to their ideal mask size. In this example, the number of ideal mask sizes available are determined by mask designers for covering different user types. This method of grouping can be grouped based on other types of data, such as grouping according to traditional 2D landmarks or grouping on principal components of face shape. The principal component analysis may be used for determining a reduced set of characteristics of facial features. An average set of 3D facial features that represent each mask size is calculated based on the groupings of mask sizes.

To size a new user, a 3D facial scan is taken or 3D data is derived from 2D images, and the fit score for the new user is calculated to each of the average faces. The mask size and type of mask selected is the mask corresponding to the average face with the lowest fit score. Additional personal preferences may be incorporated. The specific facial features could also be used to create a customized sizing based on modifying one of the available mask types.

As explained above, a facial image may be captured by a mobile computing device such as the smart phone 170. An appropriate application executed on the computing device 170 or the server 310 can provide three-dimensional relevant facial data to assist in selection of an appropriate mask. The application may use any appropriate method of facial scanning. A detailed process of facial scanning include the techniques disclosed in WO 2017000031, hereby incorporated by reference in its entirety.

One such application is an application for facial feature measuring and/or user interface sizing, which may be an application downloadable to a mobile device, such as the mobile device 170 in FIG. 1. The application, which may be stored on a computer-readable medium, such as memory/data storage 114, includes programmed instructions for processor 112 to perform certain tasks related to facial feature measuring and/or user interface evaluation. The application also includes data that may be processed by the algorithm of the automated methodology. Such data may include a data record, reference feature, and correction factors, as explained in additional detail below.

The application is executed by the processor 112, to measure user facial features using two-dimensional or three-dimensional images and to evaluate appropriate user interface sizes and types, such as from a group of standard sizes, based on the resultant measurements. The method may generally be characterized as including three or four different phases: a pre-capture phase, a capture phase, a post-capture image processing phase, and a comparison and output phase.

In some cases, the application for facial feature measuring and user interface evaluation may control the processor 112 to output a visual display that includes a reference feature on the display interface 172. The user may position the feature adjacent to their facial features, such as by movement of the camera 150. The processor may then capture and store one or more images of the facial features in association with the reference feature when certain conditions, such as alignment conditions are satisfied. This may be done with the assistance of a mirror. The mirror reflects the displayed reference feature and the user's face to the camera 150. The application then controls the processor 112 to identify certain facial features within the images and measure distances therebetween. By image analysis processing a scaling factor may then be used to convert the facial feature measurements, which may be pixel counts, to standard mask measurement values based on the reference feature. Such values may be, for example, standardized unit of measure, such as a meter or an inch, and values expressed in such units suitable for mask evaluation. Additional correction factors may be applied to the measurements.

In the pre-capture phase, the processor 112, among other things, assists the user in establishing the proper conditions for capturing one or more images for sizing processing. Some of these conditions include proper lighting and camera orientation and motion blur caused by an unsteady hand holding the computing device 170, for example.

A user may conveniently download an application for performing the automatic measuring and sizing at a user device such as a computing device 170 from a server, such as a third party application-store server, onto their computing device 170. When downloaded, such application may be stored on the computing device's internal non-volatile memory, such as RAM or flash memory.

When the user launches the application, processor 112 may prompt the user via the display interface 172 to provide user specific information, such as age, gender, weight, and height. However, processor 112 may prompt to the user to input this information at any time, such as after the user's facial features are measured. Processor 112 may also present a tutorial, which may be presented audibly and/or visually, as provided by the application to aid the user in understanding their role during the process. The prompts may also require information for user interface type, e.g., nasal or full face, etc. and of the type of device for which the user interface will be used. Also, in the pre-capture phase, the application may extrapolate the user specific information based on information already gathered by the user, such as after receiving captured images of the user's face, and based on machine learning techniques or through artificial intelligence.

When the user is prepared to proceed, which may be indicated by a user input or response to a prompt via user control/input interface, processor 112 activates an image sensor as instructed by the processor control instructions of the application. The image sensor is preferably the mobile device's forward facing camera, which is located on the same side of the mobile device as display interface 172. The camera 150 is generally configured to capture two-dimensional images. Mobile device cameras that capture two-dimensional images are ubiquitous. The present technology takes advantage of this ubiquity to avoid burdening the user with the need to obtain specialized equipment.

Around the same time the sensor/camera is activated, the processor 112, as instructed by the application, presents a capture display on the display interface 172. The capture display may include a camera live action preview, a reference feature, a targeting box, and one or more status indicators or any combination thereof In this example, the reference feature is displayed centered on the display interface and has a width corresponding to the width of the display interface 172. The vertical position of the reference feature may be such that the top edge of reference feature abuts the upper most edge of the display interface 172 or the bottom edge of reference feature abuts the lower most edge of the display interface 172. A portion of the display interface 172 will display the camera live action preview, typically showing the facial features captured by sensor/camera in real time if the user is in the correct position and orientation.

The reference feature is a feature that is known to computing device (predetermined) and provides a frame of reference to processor 112 that allows processor 112 to scale captured images. The reference feature may preferably be a feature other than a facial or anatomical feature of the user. Thus, during the image processing phase, the reference feature assists processor 112 in determining when certain alignment conditions are satisfied, such as during the pre-capture phase. The reference features may be a quick response (QR) code or known exemplar or marker, which can provide processor 112 certain information, such as scaling information, orientation, and/or any other desired information which can optionally be determined from the structure of the QR code. The QR code may have a square or rectangular shape. When displayed on display interface 172, the reference feature has predetermined dimensions, such as in units of millimeters or centimeters, the values of which may be coded into the application and communicated to processor 112 at the appropriate time. The actual dimensions of reference feature may vary between various computing devices. In some versions, the application may be configured to be a computing device model-specific in which the dimensions of reference feature, when displayed on the particular model, is already known. However, in other embodiments, the application may instruct processor 112 to obtain certain information from the device, such as display size and/or zoom characteristics that allow the processor 112 to compute the real world/actual dimensions of the reference feature as displayed on display interface 172 via scaling. Regardless, the actual dimensions of the reference feature as displayed on the display interfaces of such computing devices are generally known prior to post-capture image processing.

Along with the reference feature, the targeting box may be displayed on display interface 172. The targeting box allows the user to align certain components within capture display 172 in targeting box, which is desired for successful image capture.

The status indicator provides information to the user regarding the status of the process. This helps ensure the user does not make major adjustments to the positioning of the sensor/camera 150 prior to completion of image capture.

Thus, when the user holds display interface 172 parallel to the facial features to be measured and presents user display interface 172 to a mirror or other reflective surface, the reference feature is prominently displayed and overlays the real-time images seen by the camera/sensor and as reflected by the mirror. This reference feature may be fixed near the top of display interface 172. The reference feature is prominently displayed in this manner at least partially so that the sensor/camera can clearly see the reference feature so that processor 112 can easily the identify feature. In addition, the reference feature may overlay the live view of the user's face, which helps avoid user confusion.

The user may also be instructed by processor 112, via display interface 172, by audible instructions via a speaker of the user device 170, or be instructed ahead of time by the tutorial, to position display interface 172 in a plane of the facial features to be measured. For example, the user may be instructed to position display interface 172 such that it is facing anteriorly and placed under, against, or adjacent to the user's chin in a plane aligned with certain facial features to be measured. For example, display interface 172 may be placed in planar alignment with the sellion and supramenton. As the images ultimately captured are two-dimensional, planar alignment helps ensure that the scale of reference feature is equally applicable to the facial feature measurements. In this regard, the distance between the mirror and both of the user's facial features and the display will be approximately the same.

When the user is positioned in front of a mirror and display interface 172, which includes the reference feature, is roughly placed in planar alignment with the facial features to be measured, processor 112 checks for certain conditions to help ensure sufficient alignment. One exemplary condition that may be established by the application, as previously mentioned, is that the entirety of the reference feature must be detected within a targeting box in order to proceed. If processor 112 detects that the reference feature is not entirely positioned within targeting box, the processor 112 may prohibit or delay image capture. The user may then move their face along with display interface 172 to maintain planarity until the reference feature, as displayed in the live action preview, is located within targeting box. This helps optimized alignment of the facial features and display interface 172 with respect to the mirror for image capture.

When processor 112 detects the entirety of reference feature within targeting box, processor 112 may read the inertial motion unit (IMU) of the computing device for detection of device tilt angle. The IMU may include an accelerometer or gyroscope, for example. Thus, the processor 112 may evaluate device tilt such as by comparison against one or more thresholds to ensure it is in a suitable range. For example, if it is determined that the computing device 170, and consequently display interface 172 and user's facial features, is tilted in any direction within about ±5 degrees, the process may proceed to the capture phase. In other embodiments, the tilt angle for continuing may be within about ±10 degrees, ±7 degrees, ±3 degrees, or ±1 degree. If excessive tilt is detected a warning message may be displayed or sounded to correct the undesired tilt. This is particularly useful for assisting the user to help prohibit or reduce excessive tilt, particularly in the anterior-posterior direction, which if not corrected, could pose as a source of measuring error as the captive reference image will not have a proper aspect ratio. In some embodiments, an algorithm which accounts for tilt may be used so that the reconstruction is less sensitive to excessive tilt.

When alignment has been determined by processor 112 as controlled by the application, processor 112 proceeds into the capture phase. The capture phase preferably occurs automatically once the alignment parameters and any other conditions precedent are satisfied. However, in some embodiments, the user may initiate the capture in response to a prompt to do so.

When image capture is initiated, the processor 310 via the sensor 340 captures a number n of images, which is preferably more than one image. For example, the processor 310 via the sensor 340 may capture about 5 to 20 images, 10 to 20 images, or 10 to 15 images, etc. The quantity of images captured may be sequential such as a video. In other words, the number of images that are captured may be based on the number of images of a predetermined resolution that can be captured by sensor/camera during a predetermined time interval. For example, if the number of images sensor/camera can capture at the predetermined resolution in 1 second is 40 images and the predetermined time interval for capture is 1 second, sensor 150 will capture 40 images for processing with processor 112. The quantity of images may be user-defined, determined by artificial intelligence or machine learning of environmental conditions detected, or based on an intended accuracy target. For example, if high accuracy is required then more captured images may be required. Although, it is preferable to capture multiple images for processing, one image is contemplated and may be successful for use in obtaining accurate measurements. However, more than one image allows average measurements to be obtained. This may reduce error/inconsistencies and increase accuracy. The images may be placed by processor 112 in stored data of memory/data storage 114 for post-capture processing.

In addition, accuracy may be enhanced by images from multiple views, especially for 3D facial shapes. For such 3D facial shapes, a front image, a side profile and some images in between may be used to capture the face shape. In relation head gear size estimations, images of the sides, top, and back of the head may increase accuracy in relation to head gear. When combining landmarks from multiple views, averaging can be done, but averaging suffers from inherent inaccuracy. Some uncertainty is assigned to landmark location, and landmarks are then weighted by uncertainty during reconstruction. For example, landmarks from a frontal image will be used to reconstruct the front part of the face, and landmarks from profile shots will we used to reconstruct the sides of the head. Typically, the images will be associated with the pose of the head (angles of rotation). In this manner, it is ensured that a number of images from different views are captured. For example, if eye iris is used as the scaling features, then images where the iris is closed (e.g., when the user blinks) need to be discarded as they cannot be scaled. This is another reason to require multiple images as certain images that may not be useful may be discarded without requesting rescan.

Once the images are captured, the images are processed by processor 112 to detect or identify facial features/landmarks and measure distances between landmarks. The resultant measurements may be used to recommend an appropriate user interface size. This processing may alternatively be performed by an external device such as a server receiving the transmitted captured images and/or on the user's computing device 170. Processing may also be undertaken by a combination of the processor 112 and an external device. In one example, the recommended user interface size may be predominantly based on the user's nose width. In other examples, the recommended user interface size may be based on the user's mouth and/or nose dimensions.

The processor 112, as controlled by the application, retrieves one or more captured images from stored data. The image is then extracted by processor 112 to identify each pixel comprising the two-dimensional captured image. Processor 112 then detects certain pre-designated facial features within the pixel formation.

Detection may be performed by processor 112 using edge detection, such as Canny, Prewitt, Sobel, or Robert's edge detection, and more advanced deep neural networks (DNNs) such as Convolutional Neural Networks (CNNs) based methods for example. These edge detection techniques/algorithms help identify the location of certain facial features within the pixel formation, which correspond to the actual facial features as presented for image capture. For example, the edge detection techniques can first identify the user's face within the image and also identify pixel locations within the image corresponding to specific facial features, such as each eye and borders thereof, the mouth and corners thereof, left and right alares, sellion, supramenton, glabella and left and right nasolabial sulci, etc. The processor 112 may then mark, tag or store the particular pixel location(s) of each of these facial features. Alternatively, or if such detection by the processor 112 is unsuccessful, the pre-designated facial features may be manually detected and marked, tagged or stored by a human operator with viewing access to the captured images through a user interface of the processor 112.

Once the pixel coordinates for these facial features are identified, the application controls processor 112 to measure the pixel distance between certain of the identified features. For example, the distance may generally be determined by the number of pixels for each feature and may include scaling. For example, measurements between the left and right alares may be taken to determine pixel width of the nose and/or between the sellion and supramenton to determine the pixel height of the face. Other examples include pixel distance between each eye, between mouth corners, and between left and right nasolabial sulci to obtain additional measurement data of particular structures like the mouth. Further distances between facial features can be measured. In this example, certain facial dimensions are used for the user interface selection process.

Other methods for facial identification may be used. For example, fitting of 3D morphable models (3DMMs) to the 2D images using DNNs may be employed. The end result of such DNN methods is a full 3D surface (comprised of thousands of vertices) of the face, ears and head that may all be predicted from a single image or multiple multi-view images. Differential rendering, which involves using photometric loss to fit the model, may be applied. This minimizes the error (including at a pixel level) between a rendered version of the 3DMM and the image.

Once the pixel measurements of the pre-designated facial features are obtained, an anthropometric correction factor(s) may be applied to the measurements. It should be understood that this correction factor can be applied before or after applying a scaling factor, as described below. The anthropometric correction factor can correct for errors that may occur in the automated process, which may be observed to occur consistently from user to user. In other words, without the correction factor, the automated process, alone, may result in consistent results from user to user, but results that may lead to a certain amount of mis-sized user interfaces. Ideally the accuracy of the face landmark predictions should be able to easily distinguish between sizes of the interface. If there are only 1-2 interface sizes, then this may require an accuracy of 2-3 mm. As the number of interface sizes increases, the accuracy range is decreased to 1-2 mm or lower. The correction factor, which may be empirically extracted from population testing, shifts the results closer to a true measurement helping to reduce or eliminate mis-sizing. This correction factor can be refined or improved in accuracy over time as measurement and sizing data for each user is communicated from respective computing devices to a server where such data may be further processed to improve the correction factor. The anthropometric correction factor may also vary between the forms of user interfaces. For instance, the correction factor for a particular user seeking an FFM may be different from the correction factor when seeking a nasal mask. Such a correction factor may be derived from tracking of mask purchases, such as by monitoring mask returns and determining the size difference between a replacement mask and the returned mask.

In order to apply the facial feature measurements to user interface evaluation, whether corrected or uncorrected by the anthropometric correction factor, the measurements may be scaled from pixel units to other values that accurately reflect the distances between the user's facial features as presented for image capture. The reference feature may be used to obtain a scaling value or values. Thus, the processor 112 similarly determines the reference feature's dimensions, which can include pixel width and/or pixel height (x and y) measurements (e.g., pixel counts) of the entire reference feature. More detailed measurements of the pixel dimensions of the many squares/dots that comprise a QR code reference feature, and/or pixel area occupied by the reference feature and its constituent parts may also be determined. Thus, each square or dot of the QR code reference feature may be measured in pixel units to determine a scaling factor based on the pixel measurement of each dot and then averaged among all the squares or dots that are measured, which can increase accuracy of the scaling factor as compared to a single measurement of the full size of the QR code reference feature. However, it should be understood that whatever measurements are taken of the reference feature, the measurements may be utilized to scale a pixel measurement of the reference feature to a corresponding known dimension of the reference feature.

Once the measurements of the reference feature are taken by processor 112, the scaling factor is calculated by processor 112 as controlled by the application. The pixel measurements of reference feature are related to the known corresponding dimensions of the reference feature, e.g., the reference feature as displayed by display interface 162 for image capture, to obtain a conversion or scaling factor. Such a scaling factor may be in the form of length/pixel or area/pixelA2. In other words, the known dimension(s) may be divided by the corresponding pixel measurement(s) (e.g., count(s)).

The processor 112 then applies the scaling factor to the facial feature measurements (pixel counts) to convert the measurements from pixel units to other units to reflect distances between the user's actual facial features suitable for mask evaluation. This may typically involve multiplying the scaling factor by the pixel counts of the distance(s) for facial features pertinent for mask sizing.

These measurement steps and calculation steps for both the facial features and reference feature are repeated for each captured image until each image in the set has facial feature measurements that are scaled and/or corrected.

The corrected and scaled measurements for the set of images may then optionally be averaged or weighted by some statistical measure such as uncertainty by the processor 112 to obtain final measurements of the user's facial anatomy. Such measurements may reflect distances between the user's facial features.

In the comparison and output phase, results from the post-capture image processing phase may be directly output (displayed) to a person of interest or compared to data record(s) to obtain an automatic recommendation for a user interface size.

Once all of the measurements are determined, the results (e.g., averages) may be displayed by processor 112 to the user via display interface 172. In one embodiment, this may end the automated process. The user/patient can record the measurements for further use by the user.

Alternatively, the final measurements may be forwarded either automatically or at the command of the user to the server 310 from the computing device 170 via the communication network 308 in FIG. 3. The server 310 may execute a fit application or individuals on the server-side may conduct further processing and analysis to determine a suitable user interface or interfaces and user interface size for a particular user.

In a further embodiment, the final facial feature measurements that reflect the distances between the actual facial features of the user are compared by processor 112 to user interface size data such as in a data record. The data record may be part of the application for automatic facial feature measurements and user interface sizing. This data record can include, for example, a lookup table accessible by processor 112, which may include user interface sizes corresponding to a range of facial feature distances/values. Multiple tables may be included in the data record, many of which may correspond to a particular form of user interface and/or a particular model of user interface offered by the manufacturer.

The example process for evaluation of user interfaces identifies key landmarks from the facial image captured by the above mentioned method. In this example, initial correlation to potential interfaces involves facial landmarks including face height, nose width and nose depth. These three facial landmark measurements are collected by the application to assist in selecting the size of a compatible mask such as through the lookup table or tables. Alternatively, other data relating to facial 3D shapes may also be used for matching the derived shape data with the surfaces of the available masks as described above. For example, landmarks and any area of the face (i.e. mouth, nose etc.) can be obtained by fitting a 3D morphable model (3DMM) onto a 3D face scan of a user. This fitting process is also known as non-rigid registration or (shrink) wrapping. Once a 3DMM is registered to a 3D scan, the mask size may be determined using any number of methods, as the points and surface of the user's face are all known.

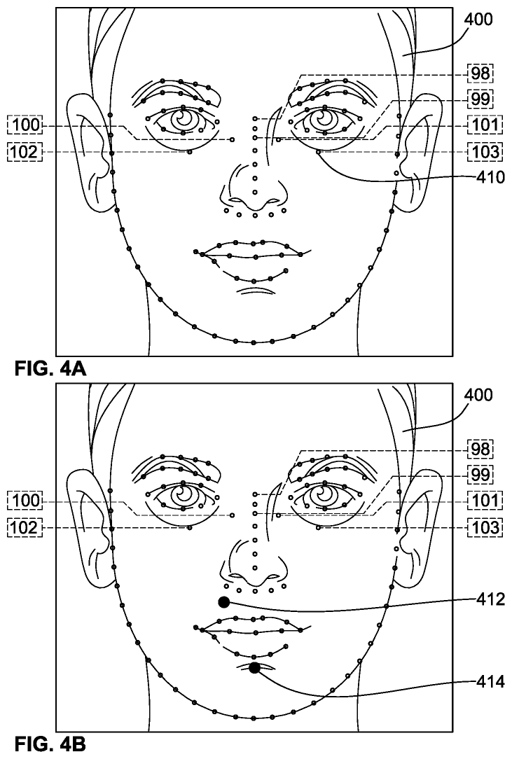

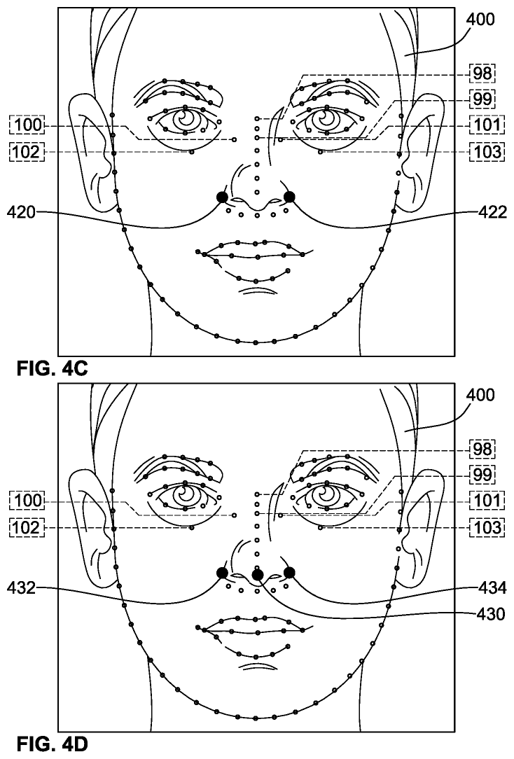

FIG. 4A is a facial image 400 such as one captured by the application described above that may be used for determining the face height dimension, the nose width dimension and the nose depth dimension. The image 400 includes a series of landmark points 410 that may be determined from the image 400 via any standardly known method. In this example, there are Standard landmark sets Open CV (68 landmark points) and some other landmarks specific to mask sizing e.g. nose, below mouth, etc. that are identified and shown on the facial image 400. In this example, the method requires seven landmarks on the facial image to determine the face height, nose width and nose depth, for mask sizing relating to the users. As will be explained, two existing landmarks may be used. The location of the three dimensions requires five additional landmarks to be identified on the image via the processing method. Based on the imaging data and/or existing landmarks, new landmarks may be determined. The two existing landmarks that will be used include a point on the sellion (nasal bridge) and a point on the nose tip. The five new landmarks required include a point on the supramenton (top of the chin), left and right alar points, and left and right alar-facial groove points.

FIG. 4B shows the facial image 400 where the face height dimension (sellion to supramenton) is defined via landmark points 412 and 414. The landmark 412 is an existing landmark point on the sellion. The landmark point 414 is a point on the supramenton. The face height dimension is determined from the distance between the landmark points 412 and 414.

FIG. 4C shows the facial image 400 with new landmark points 420 and 422 to locate the nose width dimension. This requires two new landmarks, one on each side of the nose. These are called the right and left alar points and may correspond to the right and left alare. The distance between these points provides the nose width dimension. The alar points are different, but similar to, the alar-facial groove points.

FIG. 4D shows the facial image 400 with landmark points 430, 432 and 434 to determine the nose depth dimension. A suitable landmark is available for the landmark point 430 at the nose tip. The landmark points 432 and 434 are determined at the left and right sides of the nose. The landmark points 432 and 434 are at alar-facial grooves on the left and right sides of the nose. These are similar to alar points but at the back of the nose. The examples are only one of many ways to define a plurality of landmarks. Other methods may result in more accurate estimations of anatomical measurements by using dense landmarks around regions of interest.

As explained above operational data of each respiratory device may be collected for a large population of users. This may include usage data based on when each user operates the respiratory therapy device and the duration of the operation. Thus, compliance data such as how long and often a user uses the respiratory therapy device over a predetermined period of time, the therapy pressure used and/or whether the amount and manner of use of the respiratory therapy device is consistent with a user's prescription of respiratory therapy, may be determined from the collected operational data. For example, one compliance standard may be acceptable use of the respiratory therapy device by a user over a 90 day period of time. Leak data may be determined from the operational data such as analysis of flow rate data or pressure data. Mask switching data using analysis of acoustic signals may be derived to determine whether the user is switching masks. The respiratory therapy device may be operational to determine the mask type based on an internal or external audio sensor such as the audio sensor with cepstrum analysis of the audio sensor output when the mask is being used. Alternatively, with older masks, operational data may be used to determine the type of mask through correlation of collected acoustic data to the acoustic signatures of known masks.

In this example, user input of other data may be collected via a user application executed on the computing device 170. The user application may be part of the user application that instructs the user to obtain the facial images or a separate application. This may also include subjective data obtained via a questionnaire with questions to gather data on comfort preferences, whether the user is a mouth or nose breather (for example, a question such as “Do you wake up with a dry mouth?”), and mask material preferences such as silicone, foam, textile, gel for example. For example, user input may be gathered through a user responding to subjective questions via the user application in relation to the comfort of the user interface. Other questions may relate to relevant user behavior such as sleep characteristics. For example, the subjective questions can include questions such as do you wake up with a dry mouth?, are you a mouth breather?, or what are your comfort preferences? Such sleep information may include sleep hours, how a user sleeps, and outside effects such as temperature, stress factors, etc. Subjective data may be as simple as a numerical rating as to comfort or more detailed response. Such subjective data may also be collected from a graphical user interface (GUI). For example, input data regarding leaks from a user interface that the user experienced during therapy may be collected by the user selecting parts of a user interface displayed on a graphic of the user interface on the GUI. The collected user input data may be assigned to the user database 330 in FIG. 3. The subjective input data from users may be used as an input for selection of the example mask type and size. Other subjective data may be collected related to the psychological safety of the user. For example, questions such as whether the user feels claustrophobic with that specific mask or how psychologically comfortable does the user feel wearing the mask next to their bed partner may be asked and inputs may be collected. If the answer to these questions is on the lower end indicating a negative response the system could recommend an interface from the interface database 340 that is less obtrusive such as a smaller mask than the user's existing mask, which may be a nasal cradle mask (a mask that seals to the user's face at an inferior periphery of the user's nose and leaves the user's mouth and nose bridge uncovered) or a nose-and-mouth mask that seals around the user's mouth and also at an inferior periphery of the user's nose but does not engage the nasal bridge (which may be known as an ultra-compact full face mask). Other questions around preferred sleeping position could also be factored in as well as questions around whether a user likes to move around a lot at night, and would the user prefer ‘freedom’ in the form of a tube up mask (e.g., a mask having conduit headgear) that may allow for more movement. Alternatively, if the user tends to lie still on their back or side a tube-down mask (e.g. a traditional style mask with the tube extending anteriorly and/or inferiorly from the mask proximate the user's nose or mouth) would be acceptable.

Other data sources may collect data outside of use of the respiratory therapy device that may be correlated to mask selection. This may include user demographic data such as age, gender or location; AHI severity indicating level of sleep apnea experienced by the user. Another example may be determining soft tissue thickness based on a computed tomography (CT) scan of a face. Other data may be the prescribed pressure settings for new users of the respiratory therapy device. If a user is prescribed a lower pressure such as 10 cm H2O, this may enable the user to wear a lighter mask suitable for lower pressures, resulting in greater comfort and/or less mask on the face as opposed to a full face with a very robust seal more suited for 20 cm H2O but may result in less comfort. However, if the user has a high pressure requirement e.g., 20 cm H2O the user may then be recommended a full face mask with a very robust seal.

After initial selection of a mask, the system continues to collect operational data from the respiratory therapy device 122 in FIG. 1. The collected data is added to the user database 330 in FIG. 3. The feedback from new and existing users may be used to refine recommendations for better mask options for subsequent users. For example, if operational data determines that a recommended interface has a high level of leaks, another interface type may be recommended to the user. Through a feedback loop, the selection algorithm may be refined to learn particular aspects of facial geometry that may be best suited to a particular interface. This correlation may be used to refine the recommendation of an interface to a new user with that facial geometry. The collected data and correlated interface type data may thus provide additional updating to the selection criteria for masks. Thus, the system may provide additional insights for improving selection of an interface for a user.

In addition to interface fit selection, the system may allow evaluation of interfaces in relation to respiratory therapy effectiveness and user compliance. The additional data allows optimization of the respiratory therapy based on data through a feedback loop.

Machine learning may be applied to optimize the mask evaluation process based on simulation of interface types interaction with facial data, operational data such as compliance with respiratory therapy and subjective feedback data. Such machine learning may be executed by the server 310. The mask evaluation algorithm may be trained with a training data set based on the simulation, the outputs of favorable operational results such as respiratory therapy device data and subjective data collected from users and inputs including facial shape data, user demographics, and mask sizes and types. Machine learning may be used to discover correlation between desired mask sizes and predictive inputs such as facial dimensions, user demographics, operational data from the respiratory therapy devices, and environmental conditions. Machine learning may employ techniques such as neural networks, clustering or traditional regression techniques. Test data may be used to test different types of machine learning algorithms and determine which one has the best accuracy in relation to predicting correlations.

The model for selection of an optimal interface for predicted comfort may be continuously updated by new input data from the system in FIG. 3. Thus, the model may become more accurate with greater use of the evaluation tool by more users wearing one or more of the existing interfaces and operating respiratory therapy devices.

The example application that may be executed on the user device 170 in FIG. 1 or the server 310 in FIG. 3, determines the genus of the interface and using face shape data and stored interface model data, computes a comfort score for each interface that is associated with its fit criteria. For example, an application that sizes interfaces based on facial landmarks may output a variety of different interfaces that may fit the user. Such interfaces may also be recommended by a skilled technician. In order to further evaluate these interfaces, the example application computes a comfort score using machine learning, with inputs of simulation data, objective data, and subjective data in the form of user responses to questions.

The comfort score for an interface in this example is determined by the machine learning module 314 in FIG. 3. The example machine learning model of the machine learning module 314 is trained on comfort scores reported by users based on simulation results from facial data and subjective feedback of mask comfort, and operational data from respiratory therapy devices, such as leak data. The training data set includes: (a) simulation results from mask on face simulations; (b) subjective feedback of mask comfort (from mask fitting studies); and c) operational data such as leak data from the respiratory therapy devices. The dimensional data for different mask models taken from CAD data may be calibrated with user data from fitting various interfaces to determine contact pressure (correlated with comfort score), and facial scan data to determine pressure points. For each face in the database, sizing and performance of looping (matching mask on face) is determined. The effects of pushing the mask on the face to minimize deformation to achieve a seal is also evaluated.

In this example, the simulation of an interface worn by a user includes using Finite Element Analysis (FEA) to simulate pushing an interface incrementally onto a patient's face (under ideal conditions such as minimal headgear tension required to achieve a good seal). The finite element analysis outputs include results for mask deformation, contact gaps between the skin and cushion, contact pressure/shear on the skin, skin deformation, and stress/strain in the cushion of the interface.

High contact pressure and stress/strain within the soft tissue typically correspond to discomfort. This could also be computed for conduit and headgear. Larger contact gaps between the skin and cushion may correspond to areas where leak may occur.

In this example, simulations are performed for all facial data in a patient database using a number of mask types and sizes. The facial data may be obtained by a 3D scan or one or more 2D images to create a 3D model, in some instances, a 3D morphable model may be used as a baseline model and then morphed to approximate the user's facial data. At the completion of this process very detailed simulation results were available for over 1000 faces, each with multiple interfaces types. Together with subjective data obtained from the user, the example machine learning model is trained to take any face shape as an input and output a comfort score for these interfaces. After training the model, the prediction output may be rapidly obtained once the model is trained. As the model is used, feedback data may be added to the user database 330 in FIG. 3 and the training data set may be refined to further increase the accuracy of the machine learning model.

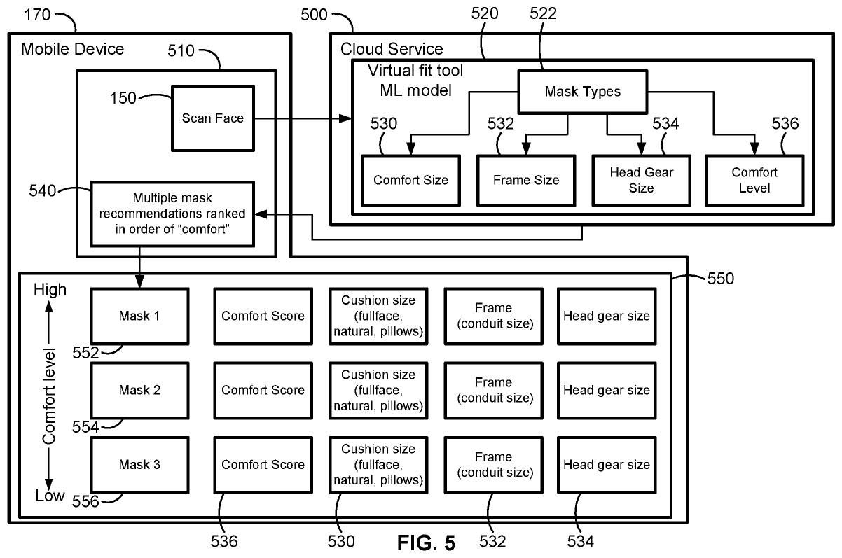

FIG. 5 shows a diagram of the overall comfort evaluation process executed between a mobile device such as the mobile device 170 in FIGS. 1 and 3, and servers in a Cloud service 500. Alternatively, the Cloud service may be any processor based device such as the network server 310 in FIG. 3. The mobile device 170 executes a comfort determination application 510 that performs a scan of the user's face using the device camera 150. As explained above, the facial data may be 2-D or 3-D data and include landmarks or more precise facial feature data. The facial data is sent to the Cloud service 500 that executes a virtual comfort application tool. In this example, the virtual comfort application tool is based on a machine learning model 520described above. The machine learning model 520 of the comfort application tool evaluates a series of interface types 522, such as different mask types, based on the simulation of available interfaces in relation to contact with facial shape. The available interface types 522 may be determined by a technician or may be automatically determined by the application 510 as fitting the face of the user based on the scanned facial data. Simulated results can be used to train a deep neural network.

The facial data is input to the machine learning model 520 of the application tool. The model simulation data of the selected interface types 522 stored on the Cloud service 500 is also input into the machine learning model 520. The machine learning model 520 outputs a cushion size 530, a frame size 532, a head gear size 534 and a comfort score 536 for each series of mask types. The resulting interface type data 540 is sent to the application 510 on the mobile device 170.

The application 510 on the mobile device 170 generates a display interface 550 that shows each of the multiple recommended mask types in order of comfort level. In this example, the display interface 550 shows three interfaces such as masks 552, 554, and 556 ranked by the predicted comfort score 536 for the individual user. The display interface 550 also shows the data for cushion size 530, conduit frame size 532, and head gear size 534 and any other relevant data such as technical data and an image of each of the masks 552, 554, and 556. The user may then select the mask 552, 554, and 556 that has the highest comfort score.

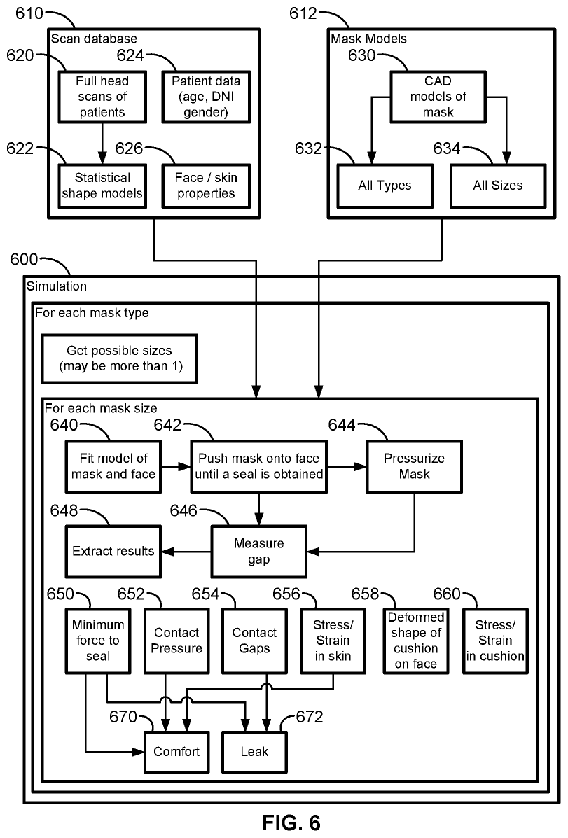

FIG. 6 shows the data flow for an interface simulation 600 executed by the application fit tool for training the machine learning model 520 for the tool in FIG. 5. The simulation 600 accesses a scan database 610 that may be stored as part of the user database 330 in FIG. 3 and a mask models database 612 that may be stored as part of the interface database 340 in FIG. 3. The scan database 610 stores the full head scan datasets 620 for different users and resulting statistical shape models 622. The shape model is also known as 3D morphable model. The scan database 610 also stores patient demographic data 624 such as age, BMI, and gender. The scan database 610 also stores facial and skin properties data 626 associated with the user. The scan database may also store other information such as landmarks with soft tissue depth, age, gender and BMI to convert surface to soft tissue properties. The data stored could also be based on anatomical scans e.g. from CT with average soft tissue thicknesses and properties.

The mask models database 612 includes dimensional data for each of the interfaces in the form of computer aided design (CAD) models 630 of all available interfaces. The CAD models 630 may be obtained from CAD data used in design and manufacturing of the interfaces. The CAD models 630 include all interfaces broken down in types of masks 632 and sizes of those masks 634.

The simulation 600 is run for each interface type and selected interface sizes (sizes with dimensions conventionally appropriate) for each face in the facial data set. For each interface size and interface type, the simulation 600 creates a finite element analysis (FEA) model 640 of the interface and the face of the user based on the provided data from the facial scan database 610 and the models database 612. The simulation then pushes the simulation of the selected mask type and size into the facial simulation 642 until a seal is obtained. The simulation then simulates the pressurization of the mask 644. After application of the simulated pressure, the simulation measures the gap between the simulated mask and the facial simulation (646).

The results of the simulation are then extracted (648). The results include the minimal force to provide the seal 650, the contact pressure 652, the contact gap 654, the stress/strain on the skin 656, the deformed shape of the cushion on the face 658, and the stress/strain on the cushion 660. These factors are then used to determine a comfort score 670 and a leak score 672. Specifically, the comfort score 670 and the leak score 672 is calculated based on the minimum force to seal, the contact pressure, and the stress/strain on the skin. Different factors may be incorporated in the score, such as the gap between cushion and face. A big gap will have a bigger leak while smaller gaps result in smaller leaks, and if there is no or close to no gap, no leaks may be expected. In terms of comfort, if contact pressure too high, the mask type may be uncomfortable and result in a lower comfort score.

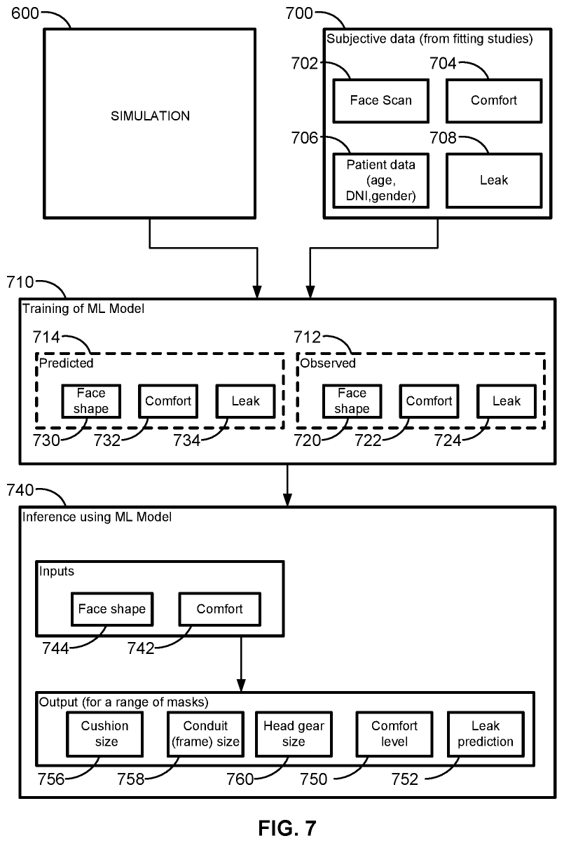

FIG. 7 shows a process for training and deploying of the machine learning model 520 accessed by the evaluation application in FIG. 5. An initial machine learning model 710 is provided with inputs such as the results of the fit simulation as explained in FIG. 6.

Additional subjective data 700 is collected from different fitting studies. The subjective data may include data from a facial scan 702, comfort data derived from subj ective queries to users 704, patient demographic data 706, and leak data 708. The comfort data is obtained from the responses to questions in the survey filled out by users as explained above in relation to different interfaces. The patient demographic data 706 is taken from the user database 330 in FIG. 3. The leak data 708 is obtained from data records of operations of respiratory therapy devices and indications of leaks such as low pressure levels, cepstrum data, and the like. Data from fitting studies and associated subjective data may be used to inform the shape model as to what features may be comfortable to a user.

The machine learning model 710 is trained based on an observed set of outputs 712 from a training data set compared with the predicted outputs 714 from the machine learning model 710. In this example, the predicted outputs 714 include facial shape data 720, comfort score data 722, and leak data 724. In this example, the facial shape data 720 is determined by scanned facial data, the comfort score data 722 is determined from the simulation in FIG. 6, and the leak data 724 is obtained from the objective and subjective data. The inputs from the simulation are provided to the machine learning model 710, which outputs a prediction of facial shape 730, comfort score 732 and leak data 734. The results are compared to the actual observed facial data 720, comfort score 722 and leak data 724 from the predicted outputs 714 of training data set. The result is a trained machine learning model 740 that accurately predicts comfort scores for a type and size of interface as well as making a leak prediction.

After training, the trained machine learning model 740 may be provided inputs including user demographic data 742 and the face shape 744 derived from facial data obtained from the facial scan of a particular user as well as mask geometry data from the mask database. The trained machine learning model 740 may then produce outputs such as a comfort score 750 and a leak prediction 752 for each type of available interface. The outputs also include a cushion size 756, conduit (frame) size 758, and headgear size 760 that are taken from the CAD data for the specific interface. Thus, the output of the machine learning model 760 is a recommendation of a specific mask type and mask size as well as the corresponding comfort level and leak prediction for the particular mask. There may be one or more predicted masks which will be an output as it may be a “close call” for some users. The output is determined by running the particular user's face shape and demographic information through the trained model 760 to determine which mask type and size would result in the best comfort level with the least amount of leak.

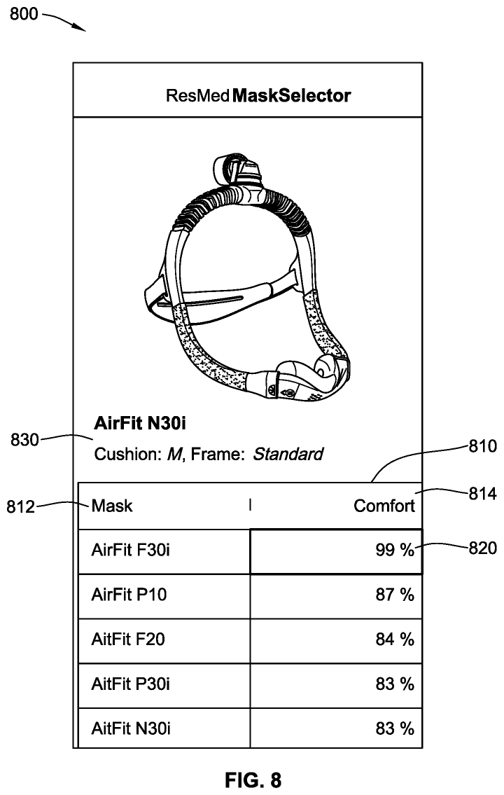

FIG. 8 shows a screen image 800 of the display interface 550 in FIG. 5 generated by the application 510 on the mobile device 170. The display interface 800 includes a table 810 that ranks all of the types of masks that fit the user. The table 810 orders the different masks according to the comfort score determined by the comfort application 510. The table 810 thus includes a mask name column 812 and a comfort score column 814. A top entry 820 is the most recommended mask for maximizing comfort based on the determined comfort score. The top entry 820 may be highlighted in a color to emphasize the selection. The other masks that may fit are ranked in the table 810 according to comfort score.

In order to assist the user, the application displays an information field 830 relating to the top ranked interface. The information field 830 may include the model number, the size of the cushion, and the type of frame associated with the mask. An image 832 of the top ranked mask may be displayed to assist the user.

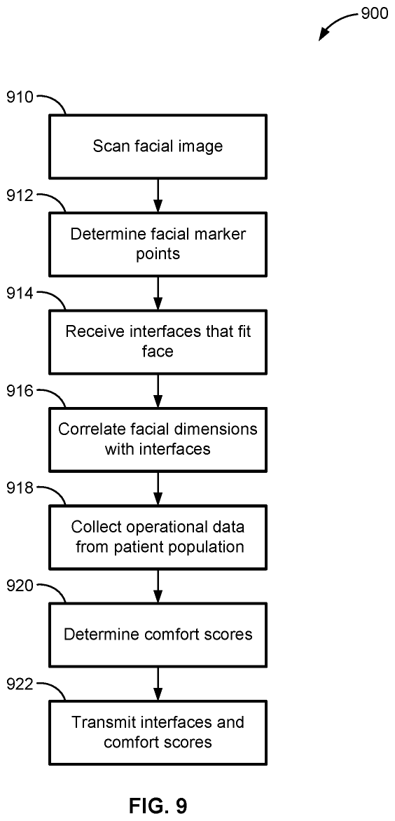

The operation of the example comfort determination application 510 shown in FIG. 5, which may be controlled on the example server and a user device, will now be described with reference to FIG. 1 in conjunction with the flow diagram shown in FIG. 9. The flow diagram in FIG. 9 is representative of example machine readable instructions for implementing the application to evaluate interfaces for a specific face of a user. In this example, the machine readable instructions comprise an algorithm for execution by: (a) a processor, (b) a controller, and/or (c) one or more other suitable processing device(s). The algorithm may be embodied in software stored on tangible media such as, for example, a flash memory, a CD-ROM, a floppy disk, a hard drive, a digital video (versatile) disk (DVD), or other memory devices, but persons of ordinary skill in the art will readily appreciate that the entire algorithm and/or parts thereof could alternatively be executed by a device other than a processor and/or embodied in firmware or dedicated hardware in a well-known manner (e.g., it may be implemented by an application specific integrated circuit (ASIC), a programmable logic device (PLD), a field programmable logic device (FPLD), a field programmable gate array (FPGA), discrete logic, etc.). For example, any or all of the components of the interfaces could be implemented by software, hardware, and/or firmware. Also, some or all of the machine readable instructions represented by the flowchart of FIG. 9 may be implemented manually. Further, although the example algorithm is described with reference to the flowcharts illustrated in FIG. 9, persons of ordinary skill in the art will readily appreciate that many other methods of implementing the example machine readable instructions may alternatively be used. For example, the order of execution of the blocks may be changed, and/or some of the blocks described may be changed, eliminated, or combined.

In this example, facial data for a user is first collected through the scan from the user device (910). The routine then determines facial marker points (912). Data from the interfaces that fit the facial marker points are obtained (914). The facial dimensions are matched with contact points on the selected interfaces (916). Operational data from the user population is collected such as leak data and comfort data related to interfaces (918). The comfort scores for each of the selected interfaces are then determined (920). The selections and comfort scores are then transmitted to the user device (922).

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method to evaluate an interface to be worn on a face of a user of a respiratory therapy device, the method comprising:
storing a facial image of the user in a storage device;
determining facial features of the user based on the facial image;
storing a plurality of facial feature data from a user population and a corresponding plurality of interface dimensional data from a plurality of interfaces used by the user population in one or more databases;
storing operational data of respiratory therapy devices used by the user population with the plurality of interfaces in one or more databases;
determining a comfort score for the interface via an evaluation tool, the evaluation tool determining the comfort score based on the facial features of the user, the output of a simulator simulating the interface on the plurality of facial feature data, and the operational data; and
displaying the comfort score on a display.

2. The method of claim 1, wherein the evaluation tool includes a machine learning model outputting the comfort score based on the facial image data and dimensional data of the interface.

3. The method of claim 2, further comprising training the machine learning model by comparing comfort scores determined from the simulator simulating the plurality of interfaces based on the interface dimensional data worn on faces of the user population based on the facial feature data, with comfort scores provided from the user population.

4. The method of claim 3, wherein the comfort scores provided from the user population are determined based on at least one of operational data of the respiratory therapy devices, the facial features data, or subjective responses of the user population derived from answers of a survey.

5. The method of claim 2, wherein the simulator models the plurality of interfaces worn on faces of the user population with finite element analysis.

6. The method of claim 2, wherein the dimensional data of the interfaces is computer aided design (CAD) data.

7. The method of claim 2, wherein the simulator simulates pushing the interfaces into the simulated faces until a seal is between the simulated faces and the simulated interface is obtained, the pressurization of the simulated interfaces, and a resulting gap between the simulated interfaces and the simulated faces.

8. The method of claim 2, wherein the simulator outputs interface deformation, contact gaps between skin of the simulated faces and cushions of the interfaces, contact pressure/shear on skin of the simulated face, skin deformation of the simulated faces, and stress/strain in the cushions of the interfaces.

9. The method of claim 1, wherein the selected interface is one of the plurality of interfaces and one of a plurality of sizes of each of the plurality of interfaces.

10. The method of claim 9, wherein the displaying includes displaying a subset of interfaces selected from the plurality of interfaces that fit the face of the user and associated comfort scores.

11. The method of claim 1, wherein the evaluation tool accepts demographic data of the user to determine the comfort score.

12. The method of claim 1, wherein the operational data from the respiratory therapy devices includes data to determine leaks in the operation of the respiratory therapy devices.

13. The method of claim 1, further comprising scanning the face of the user via a mobile device including a camera to provide the facial image.

14. The method of claim 13, wherein the mobile device includes a depth sensor, and wherein the camera is a 3D camera, and wherein the facial features are three-dimensional features derived from a meshed surface derived from the facial image.

15. The method of claim 1, wherein the facial image is a two-dimensional image including landmarks, wherein the facial features are three-dimensional features derived from the landmarks.

16. The method of claim 1, wherein the facial image is one of a plurality of two-dimensional facial images, and wherein the facial features are three-dimensional features derived from a 3D morphable model adapted to match the facial images.

17. The method of claim 1, wherein the facial image includes landmarks relating to at least one facial dimension including least one of face height, nose width, and nose depth.

18. The method of claim 1, further comprising determining a predicted leak of the interface via the evaluation tool.

19. A system for evaluating a selected interface worn by a user using a respiratory therapy device, the system comprising:
a storage device for storing facial image data of the user;
one or more databases for storing:
a plurality of facial feature data from a user population and a corresponding plurality of interface dimensional data from a plurality of interfaces used by the user population;
operational data of respiratory therapy devices used by the user population with the plurality of interfaces;
a facial comfort interface evaluation tool coupled to the storage device, the evaluation tool outputting a comfort score of the interface based on analysis of the facial image data of the user, the output of a simulator simulating the interface on the plurality of facial feature data, and the operational data; and
a display to display the comfort score of the interface.

20. A method of training a machine learning model to output a comfort score for an interface worn by a user, method comprising:
collecting dimensional data for a plurality of interfaces for a respiratory therapy device;
collecting facial data from a plurality of faces of users wearing the plurality of interfaces;
determining a comfort score for each of the plurality of interfaces worn by users;
simulating the plurality of interfaces worn on faces of the user population based on dimensional data of the plurality of interfaces and facial dimensional data derived from the facial data of the plurality of faces;
creating a training data set of the dimensional data of the plurality of interfaces and the facial dimension data; and
adjusting the machine learning model by providing the training data set and the simulation to predict a comfort score for each face and worn interface, and comparing the predicted comfort score with the associated determined comfort score.
